(19) United States
(12) Patent Application Publication
(10) Pub. No.: US 2026/0244095 A1
(43) Pub. Date: Aug. 20, 2026

(54) PHOTOSENSITIVE RESIN COMPOSITION, METHOD FOR PRODUCING RESIST PATTERN FILM, AND METHOD FOR PRODUCING PLATED FORMED PRODUCT

(71) Applicant: JSR CORPORATION, Tokyo (JP)

(72) Inventors: Takuya TOMITA, Minato-ku, Tokyo (JP); Hisanori AKIMARU, Minato-ku, Tokyo (JP)

(73) Assignee: JSR CORPORATION, Tokyo (JP)

(21) Appl. No.: 19/160,505

(22) PCT Filed: Mar. 7, 2024
(86) PCT No.: PCT/JP2024/008759

§ 371 Date: Aug. 28, 2025

(30) Foreign Application Priority Data
Mar. 15, 2023 (JP) 2023-040524

(51) Int. Cl. G03F 7/004; C07C 309/02; C07C 311/48; C07C 381/12; C07D 333/46; C08F 22/10; C25D 5/02; G03F 7/039; H10W 70/05

(52) U.S. Cl. CPC G03F 7/0045; C07C 309/02; C07C 311/48; C07C 381/12; C07D 333/46; C08F 22/10; C25D 5/022; G03F 7/0397; H10W 70/05

(57) ABSTRACT

One aspect of the present invention relates to a photosensitive resin composition. The photosensitive resin composition contains a polymer (A) having an acid-dissociable group, a photoacid generator (B), a solvent (C), and a compound (D). The compound (D) is a compound which is decomposed by the action of an acid generated from the photoacid generator (B) to become a compound (d), and is a compound having a group which is decomposed by the action of an acid to generate a compound (d′) (however, the compound (D) is different from the polymer (A), and the compound (d) and the compound (d′) are non-basic compounds), and the compound (d′) has a boiling point of 65° C. or higher.

### TECHNICAL FIELD

One aspect of the present invention relates to a photosensitive resin composition, a method for producing a resist pattern film, and a method for producing a plated formed product.

### BACKGROUND ART

Performance enhancement of mobile devices such as smartphones and tablet terminals is performed by packaging semiconductor chips having different functions using a high-density packaging technology such as a fan-out wafer level package (FO-WLP), a fan-out panel level package (FO-PLP), a through silicon via (TSV), or a silicon interposer.

In such a packaging technology, wiring lines and bumps used for electrical connection between semiconductor chips are also increasing in density. Therefore, a fine and high-density resist pattern film used for forming wiring lines and bumps is also required.

Usually, wiring lines and bumps are plated formed products, and are produced by applying a photosensitive resin composition onto a substrate having a metal film such as a copper film to form a resist coating film, exposing and developing the resist coating film using a mask to form a resist pattern film, and performing a plating treatment on the substrate using the resist pattern film as a mold.

As described above, since a resist pattern film is formed on a metal film, and then a plating treatment is performed, the photosensitive resin composition is required to improve, for example, the adhesiveness between the resist pattern film and the metal film, the rectangularity of the resist pattern shape that affects the shape of a plated formed product, and a standing wave (a phenomenon in which a resist pattern becomes rough (wave-shaped) due to an interference wave caused by interference between incident light and reflected light from a substrate; it is also referred to as “line edge roughness”). One of the factors that affect the adhesiveness of a plated formed product is a trailing shape at the interface between a metal film and a resist pattern film.

In particular, in order to improve a standing wave, a method of adding a high boiling point solvent is known, but insufficient resolution or a pattern shape defect such as a decrease in rectangularity may occur. For example, Patent Literatures 1 to 3 propose a radiation-sensitive resin composition (photosensitive resin composition) achieving both improvement of a standing wave and insufficient resolution and/or a pattern shape defect in such a trade-off relationship.

### CITATION LIST

### Patent Literature

Patent Literature 1: JP 2011-53622 A
Patent Literature 2: JP 2011-173885 A
Patent Literature 3: JP 2006-323011 A

### SUMMARY OF INVENTION

### Technical Problem

However, in a resist pattern film that has been required to be further thinned and miniaturized in recent years, there has been room for improvement in the balance between trailing and a standing wave in the photosensitive resin composition as described in Patent Literatures 1 to 3.

One aspect of the present invention provides a photosensitive resin composition capable of producing a resist pattern film excellent in balance between a standing wave and a trailing shape, and a method for producing the resist pattern film and a plated formed product formed from the resist pattern film.

### Solution to Problem

The present inventors conducted studies in order to solve the above problems. As a result, according to the following aspect examples, it has been found that the above problem can be solved, and the present invention has been completed. That is, the aspect examples of the present invention relate to, for example, the following [1] to [18].

[1]

A photosensitive resin composition containing a polymer (A) having an acid-dissociable group, a photoacid generator (B), a solvent (C), and a compound (D), in which
the compound (D) is a compound which is decomposed by an action of an acid generated from the photoacid generator (B) to become a compound (d) and is a compound having a group which is decomposed by an action of an acid to generate a compound (d′) (provided that the compound (D) is different from the polymer (A), and the compound (d) and the compound (d′) are non-basic compounds), and
the compound (d′) has a boiling point of 65° C. or higher.
   [2]

The photosensitive resin composition according to [1], in which the compound (D) has a group represented by a formula (i) below:

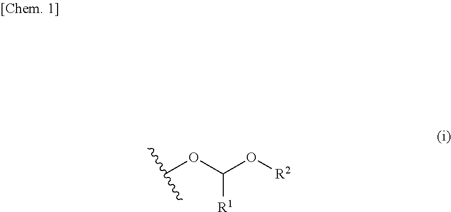

in the formula (i), R¹ represents an alkyl group having 1 to 3 carbon atoms, R² represents an alkyl group having 2 to 10 carbon atoms, a cycloalkyl group having 3 to 20 carbon atoms, an aryl group having 6 to 10 carbon atoms, a group obtained by combining these groups, or a group in which these groups are linked via an ether bond, or represents a cyclic structure represented by bonding carbon atoms contained in these groups of R¹ and R² to each other.
   [3]

A photosensitive resin composition containing a polymer (A) having an acid-dissociable group, a photoacid generator (B), a solvent (C), and a compound (D), in which
the compound (D) is a compound which is decomposed by an action of an acid generated from the photoacid generator (B) to become a compound (d) and is a compound which is decomposed by an action of an acid to generate a compound (d′) (provided that the compound (D) is different from the polymer (A), and the compound (d) and the compound (d′) are non-basic compounds), and
the compound (D) has a group represented by a formula (i) below:

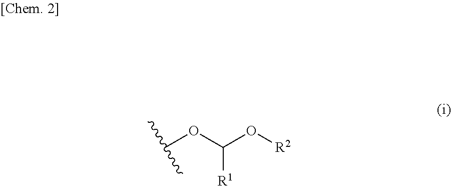

in the formula (i), R¹ represents an alkyl group having 1 to 3 carbon atoms, R² represents an alkyl group having 2 to 10 carbon atoms, a cycloalkyl group having 3 to 20 carbon atoms, an aryl group having 6 to 10 carbon atoms, a group obtained by combining these groups, or a group in which these groups are linked via an ether bond, or represents a cyclic structure represented by bonding carbon atoms contained in these groups of R¹ and R² to each other.
   [4]

The photosensitive resin composition according to any one of [1] to [3], in which the compound (D) is a compound represented by any of formulae (I) to (IV) below:

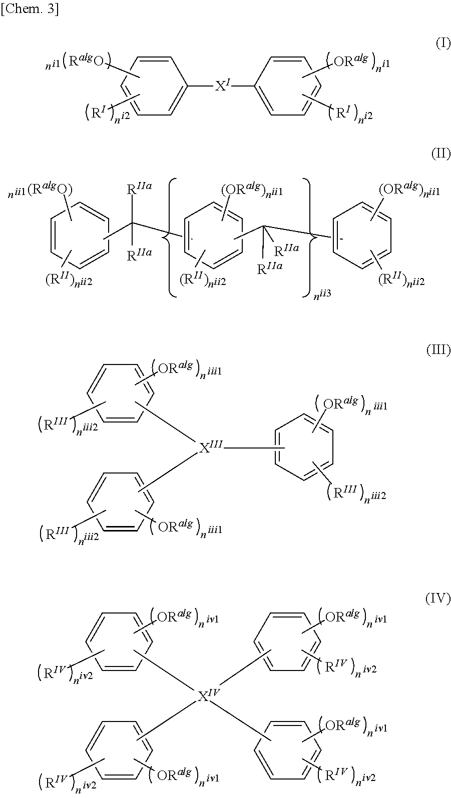

in the formulae (I) to (IV),
R^{alg}'s independently represent a hydrogen atom or a group that generates the compound (d′);
R^{I} to R^{IV} each independently represent an alkyl group having 1 to 4 carbon atoms, a cycloalkyl group having 3 to 10 carbon atoms, an aryl group having 6 to 10 carbon atoms, or a halogen atom;
R^{IIa}'s independently represent a hydrogen atom or an alkyl group having 1 or 2 carbon atoms;
nⁱ¹ to n^{iv1} and nⁱⁱ³ each independently represent an integer of 1 or 2;
nⁱ² to n^{iv2} each independently represent an integer of 0 to 2;
X^{I} represents a single bond or a structure represented by formulae (I-1) to (I-3) below:

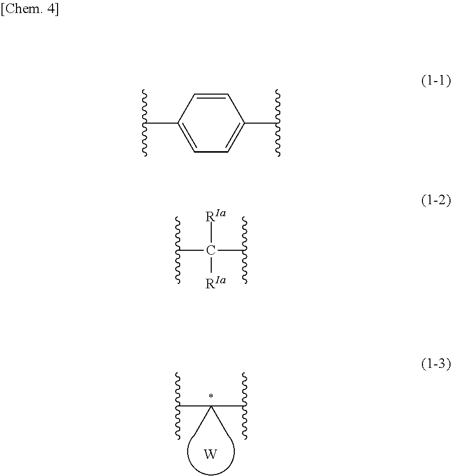

in the formula (I-2), R^{Ia} represents a hydrogen atom, an alkyl group having 1 to 10 carbon atoms, a cycloalkyl group having 3 to 10 carbon atoms, an aryl group having 6 to 10 carbon atoms, a halogen atom, or a group represented by a formula (I-2a) below:

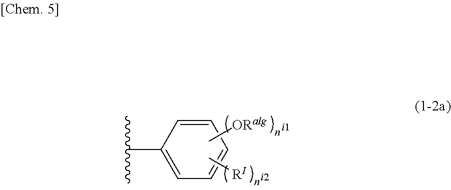

symbols in the formula (I-2a) indicate similar meanings to the same symbols in the formula (I),
in the formula (I-3), W represents a structure represented by a formula (I-3a) or (I-3b) below:

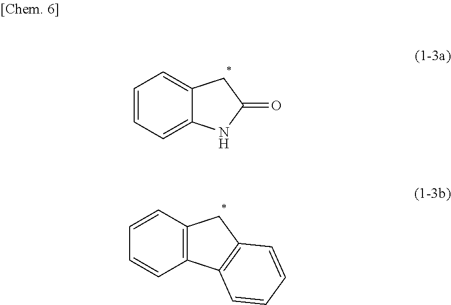

in the formulae (I-3a) and (I-3b), a carbon atom with * indicates the same carbon atom as * in the formula (I-3),
X^{III} represents a structure represented by a formula (III-1) or (III-2) below:

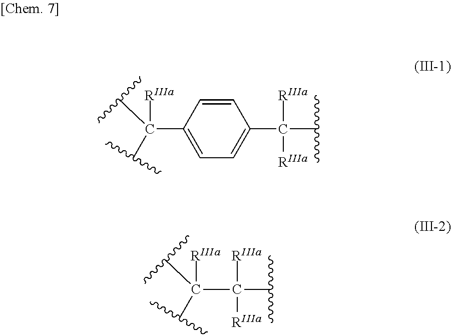

in the formulae (III-1) and (III-2), R^{IIIa} represents a substituent similar to R^{IIa} in the formula (II),
X^{IV} represents a structure represented by formulae (IV-1) to (IV-3) below:

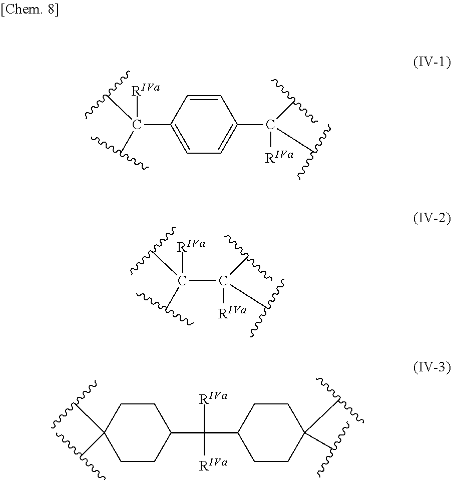

in the formulae (IV-1) to (IV-3), R^{IVa} represents a substituent similar to R^{IIa} in the formula (II).
   [5]

The photosensitive resin composition according to [4], in which the compound (D) is a compound represented by the formula (I) or (IV).

[6]

The photosensitive resin composition according to [4] or [5], in which the compound (D) is a compound represented by a formula (I) in which the X^{I} has a structure represented by the formula (I-2) or a compound represented by a formula (IV) in which the X^{IV} has a structure represented by the formula (IV-2).

[7]

The photosensitive resin composition according to any one of [1] to [6], in which the compound (D) is a compound having two or more groups that generate the compound (d′) on average in one molecule.

[8]

The photosensitive resin composition according to any one of [1] to [7], in which a content of the compound (D) is 1 to 40 parts by mass with respect to 100 parts by mass of the compound (A).

[9]

The photosensitive resin composition according to any one of [1] to [8], in which the polymer (A) having an acid-dissociable group has a structural unit represented by a formula (a1-1) having an acid-dissociable group below:

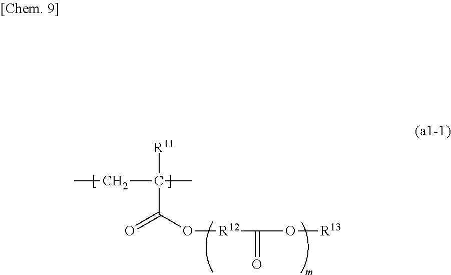

in the formula (a1-1), R¹¹ is a hydrogen atom, an alkyl group having 1 to 10 carbon atoms, or a group obtained by substituting at least one hydrogen atom in the alkyl group with a halogen atom, an aryl group, a hydroxy group, or an alkoxy group, R¹² is a divalent organic group having 1 to 10 carbon atoms, R¹³ is an acid-dissociable group, and m is an integer of 0 to 10.
   [10]

The photosensitive resin composition according to any one of [1] to [9], further containing a compound (E1) which is decomposed by an action of an acid to generate a primary or secondary amine.

[11]

The photosensitive resin composition according to any one of [1] to [10], in which the photoacid generator (B) is a compound represented by a formula (b1) or (b2) below:

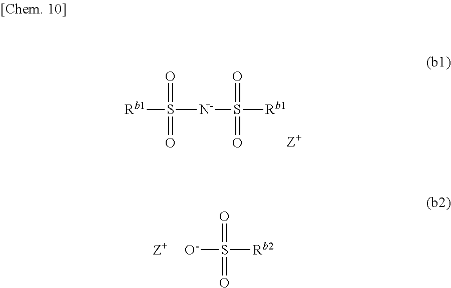

in the formulae (b1) and (b2), Z⁺ represents an onium cation, R^{b1} and R^{b2} each independently represent an alkyl group having 1 to 10 carbon atoms, a cycloalkyl group, an aryl group, or a group obtained by combining these groups, and the alkyl group, the cycloalkyl group, and the aryl group are unsubstituted or some or all hydrogen atoms in these groups are substituted with a fluorine atom, provided that a case where two R^{b1}'s represent a cyclic structure represented by bonding carbon atoms contained in these groups to each other is included.
   [12]

The photosensitive resin composition according to any one of [1] to [11], further containing an alkali-soluble resin.

[13]

The photosensitive resin composition according to any one of [1] to [12], in which the solvent (C) contains at least one selected from propylene glycol monomethyl ether acetate, 3-methoxybutyl acetate, and propylene glycol monomethyl ether.

[14]

A method for producing a resist pattern film, the method including:
a step (1) of forming a resin film of the photosensitive resin composition according to any one of [1] to [13] on a metal film of a substrate having the metal film;
a step (2) of exposing the resin film to light; and
a step (3) of developing the resin film after exposure.
   [15]

The method for producing a resist pattern film according to [14], in which the resist pattern film has a thickness of 0.5 to 100 μm.

[16]

A method for producing a plated formed product, the method including a step of performing a plating treatment using a resist pattern film produced by the method for producing a resist pattern film according to [14] or [15] as a template.

[17]

The method for producing a plated formed product according to [16], further including a step of performing an ashing treatment before the step of performing the plating treatment.

[18]

The method for producing a plated formed product according to [16] or [17], wherein the plating treatment is one selected from copper, nickel, tin, and gold plating treatments.

### Advantageous Effects of Invention

According to one aspect of the present invention, it is possible to provide a photosensitive resin composition capable of producing a resist pattern film excellent in balance between a standing wave and a trailing shape, and a method for producing the resist pattern film and a plated formed product formed from the resist pattern film.

### BRIEF DESCRIPTION OF DRAWINGS

FIG. 1 is a view illustrating a method for evaluating a standing wave in examples.

FIG. 2 is a view illustrating a method for evaluating trailing in examples.

### DESCRIPTION OF EMBODIMENTS

Hereinafter, embodiments for carrying out the present invention will be described.

### [Photosensitive Resin Composition]

The photosensitive resin composition according to an aspect of the present invention (hereinafter also referred to as “present composition”) contains a polymer (A) having an acid-dissociable group, a photoacid generator (B), a solvent (C), and a compound (D).

The compound (D) is a compound which is decomposed by the action of an acid generated from the photoacid generator (B) to become a compound (d), and is a compound having a group which is decomposed by the action of an acid to generate a compound (d′). Furthermore, the compound (D) satisfies either of the following requirements (D1) and (D2).
(D1): The boiling point of the compound (d′) is 65° C. or higher.
(D2): The compound (D) has a group represented by a formula (i) below.

Each component will be described below.

### <Polymer (A)>

The polymer (A) has an acid-dissociable group. The acid-dissociable group is a group that can be dissociated by the action of an acid generated from the photoacid generator (B) described later. As a result of the dissociation, an acidic functional group such as a carboxy group or a phenolic hydroxy group is generated in the polymer (A). As a result, the solubility of the polymer (A) in an alkaline developer changes, and the present composition enables the formation of a positive resist pattern film.

The polymer (A) has an acidic functional group protected by an acid-dissociable group. Examples of the acidic functional group include a carboxy group and a phenolic hydroxy group. Examples of the polymer (A) include a (meth)acrylic resin in which a carboxy group is protected by an acid-dissociable group, and a polyhydroxystyrene resin in which a phenolic hydroxy group is protected by an acid-dissociable group.

The weight average molecular weight (Mw) of the polymer (A) in terms of polystyrene measured by gel permeation chromatography (GPC) is usually 1,000 to 500,000, preferably 3,000 to 300,000, more preferably 10,000 to 100,000, and still more preferably 20,000 to 60,000.

The ratio of the Mw of the polymer (A) to the number average molecular weight (Mn) in terms of polystyrene measured by gel permeation chromatography (molecular weight distribution; Mw/Mn) is usually 1 to 5, preferably 1 to 3. In the present description, Mw and Mn of the polymer can be measured by the method described in examples below.

The present composition can contain one type or two or more types of polymers (A).

The content ratio of the polymer (A) in the present composition is usually 60 to 99.5 mass %, preferably 63 to 99 mass %, and more preferably 65 to 98 mass % in 100 mass % of the solid content of the present composition. The solid content refers to all components other than a solvent (C) and a quencher (E) described later.

<<Structural Unit (a1)>>

The polymer (A) preferably has a structural unit (a1) having an acid-dissociable group.

Examples of the structural unit (a1) include a structural unit represented by a formula (a1-1) below and a structural unit represented by a formula (a1-2) below, and a structural unit represented by a formula (a1-1) below is particularly preferable.

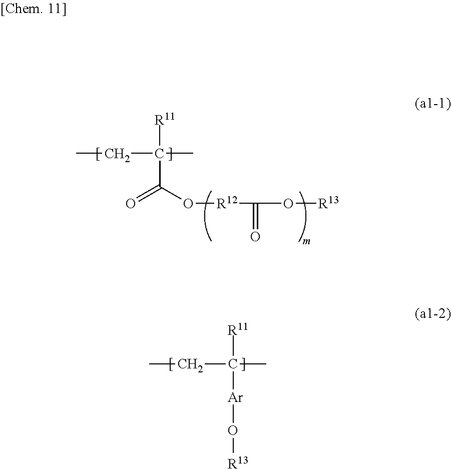

In the formulae (a1-1) and (a1-2), R¹¹ is a hydrogen atom, an alkyl group having 1 to 10 carbon atoms, or a group obtained by substituting at least one hydrogen atom in the alkyl group with another group such as a halogen atom, an aryl group, a hydroxy group, or an alkoxy group (hereinafter also referred to as “substituted alkyl group”), and R¹³ is an acid-dissociable group.

In the formula (a1-1), R¹² is a divalent organic group having 1 to 10 carbon atoms, and m is an integer of 0 to 10, preferably an integer of 0 to 5, and more preferably an integer of 0 to 3.

In the formula (a1-2), Ar is an arylene group having 6 to 10 carbon atoms.

Examples of the alkyl group having 1 to 10 carbon atoms include a methyl group, an ethyl group, a n-propyl group, an isopropyl group, a n-butyl group, a pentyl group, and a decyl group.

Examples of the halogen atom include a fluorine atom and a bromine atom, examples of the aryl group include a phenyl group, and examples of the alkoxy group include a methoxy group and an ethoxy group.

Examples of the divalent organic group having 1 to 10 carbon atoms include alkanediyl groups having 1 to 10 carbon atoms such as a methylene group, an ethylene group, a propane-1,3-diyl group, a propane-1,2-diyl group, and a decane-1,10-diyl group; and a group obtained by substituting at least one hydrogen atom in the alkanediyl group with another group such as a halogen atom, an aryl group, a hydroxy group, or an alkoxy group.

Examples of the arylene group having 6 to 10 carbon atoms include a phenylene group, a methylphenylene group, and a naphthylene group.

Specific examples of the acid-dissociable group include an acid-dissociable group represented by a formula (g1) below or a benzyl group, and an acid-dissociable group represented by a formula (g1) below is preferable.

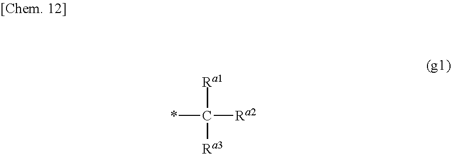

In the formula (g1), [symbol] represents a bond, R^{a1} to R^{a3} are each independently an alkyl group, a cycloalkyl group, or a group obtained by substituting at least one hydrogen atom in the alkyl group or the cycloalkyl group with another group such as a halogen atom, an aryl group, a hydroxy group, or an alkoxy group, and two groups of R^{a1} to R^{a3} may be bonded to each other to form an alicyclic structure.

Examples of the alkyl group of R^{a1} to R^{a3} include alkyl groups having 1 to 10 carbon atoms, and specific examples thereof include groups similar to those exemplified in R¹¹.

Examples of the cycloalkyl group of R^{a1} to R^{a3} include monocyclic saturated cyclic hydrocarbon groups such as a cyclobutyl group, a cyclopentyl group, a cyclohexyl group, a cycloheptyl group, and a cyclooctyl group; monocyclic unsaturated cyclic hydrocarbon groups such as a cyclobutenyl group, a cyclopentenyl group, and a cyclohexenyl group; polycyclic saturated cyclic hydrocarbon groups such as a norbornyl group, an adamantyl group, a tricyclodecyl group, and a tetracyclododecyl group.

Examples of the alicyclic structure formed by two groups of R^{a1} to R^{a3} include monocyclic saturated cyclic hydrocarbon structures such as cyclobutyl, cyclopentyl, cyclohexyl, cycloheptyl, and cyclooctyl; monocyclic unsaturated cyclic hydrocarbon structures such as cyclobutenyl, cyclopentenyl, and cyclohexenyl; polycyclic saturated cyclic hydrocarbon structures such as norbornyl, adamantyl, tricyclodecyl, and tetracyclododecyl.

As the acid-dissociable group represented by the formula (g1), groups represented by formulae (g1-1) to (g1-5) below are preferable, and a group represented by a formula (g1-5) below is particularly preferable.

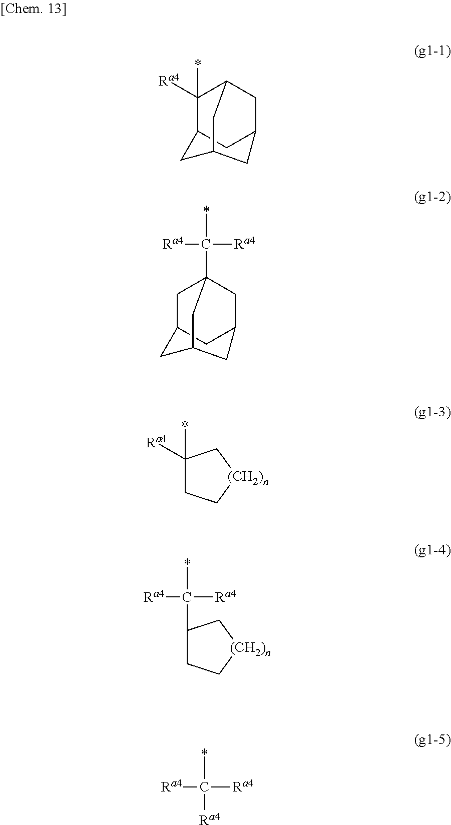

In the formulae (g1-1) to (g1-5), * represents a bond, R^{a4} is independently an alkyl group having 1 to 10 carbon atoms, and n is an integer of 1 to 4.

Each alicyclic structure in the formulae (g1-1) to (g1-4) may have one or two or more substituents such as an alkyl group having 1 to 10 carbon atoms, a halogen atom, a hydroxy group, and an alkoxy group.

Examples of the structural unit (a1) include, in addition to the structural units represented by the formulae (a1-1) and (a1-2), structural units having an acetal-based acid-dissociable group described in, for example, JP 2005-208366 A, JP 2000-194127 A, JP 2000-267283 A, and JP 2004-348106 A; structural units having a sultone ring described in, for example, JP 2013-101321 A; and structural units having a crosslinked acid-dissociable group described in, for example, JP 2000-214587 A and JP 2000-199960 A. The structural units described in the above-mentioned publications shall be described in the present description.

The polymer (A) can have one type or two or more types of structural units (a1).

The content ratio of the structural unit (a1) in the polymer (A) is usually 10 to 60 mol %, preferably 15 to 55 mol %, and more preferably 20 to 50 mol %. The content ratio of each structural unit in the polymer (A) is a value when the total of all structural units constituting the polymer (A) is taken as 100 mol %. Each of the structural units is usually derived from a monomer during synthesis of the polymer (A). The content ratio of each structural unit can be measured by, for example, ¹H-NMR.

<<Structural Unit (a2)>>

The polymer (A) can further have a structural unit (a2) having a group that promotes solubility in an alkaline developer. By adjusting the content ratio of the structural unit (a2) in the polymer (A), it tends to be possible to adjust the lithographic properties such as resolution, sensitivity, and depth of focus of a resin film to be formed from the present composition.

Examples of the structural unit (a2) include a structural unit having at least one group or structure selected from a phenolic hydroxy group, a carboxy group, an alcoholic hydroxy group, a lactone structure, a cyclic carbonate structure, a sultone structure, and a fluoroalcohol structure (however, those corresponding to the structural unit (a1) are excluded). Among them, a structural unit having a phenolic hydroxy group is preferable because a resist pattern film that is strong against pressing from plating at the time of forming a plated formed product can be formed.

Examples of the structural unit having a phenolic hydroxy group include a structural unit derived from a monomer having a hydroxyaryl group such as 2-hydroxystyrene, 4-hydroxystyrene, 4-isopropenylphenol, 4-hydroxy-1-vinylnaphthalene, 4-hydroxy-2-vinylnaphthalene, or 4-hydroxyphenyl (meth)acrylate.

Examples of the hydroxyaryl group include hydroxyphenyl groups such as a hydroxyphenyl group, a methylhydroxyphenyl group, a dimethylhydroxyphenyl group, a dichlorohydroxyphenyl group, a trihydroxyphenyl group, and a tetrahydroxyphenyl group; and hydroxynaphthyl groups such as a hydroxynaphthyl group and a dihydroxynaphthyl group.

Examples of the structural unit having a carboxy group include structural units derived from a monomer such as (meth)acrylic acid, crotonic acid, maleic acid, fumaric acid, cinnamic acid, 2-carboxyethyl (meth)acrylate, 2-carboxypropyl (meth)acrylate, or 3-carboxypropyl (meth)acrylate, and structural units described in JP 2002-341539 A.

Examples of the structural unit having an alcoholic hydroxy group include structural units derived from a monomer such as 2-hydroxyethyl (meth)acrylate or 3-(meth)acryloyloxy-4-hydroxytetrahydrofuran, and structural units described in JP 2009-276607 A.

The polymer (A) can have one type or two or more types of structural units (a2).

When the polymer (A) has the structural unit (a2), the content ratio of the structural unit (a2) in the polymer (A) is usually 10 to 80 mol %, preferably 20 to 65 mol %, and more preferably 25 to 60 mol %. When the content ratio of the structural unit (a2) is within the above range, the dissolution rate in an alkaline developer can be increased, and as a result, the resolution in a thick film of the present composition can be improved.

The polymer (A) can have the structural unit (a2) in a polymer the same as or different from a polymer having the structural unit (a1), and preferably has the structural units (a1) and (a2) in the same polymer.

<<Structural Unit (a3)>>

The polymer (A) may further have another structural unit (a3) other than the structural units (a1) and (a2). Examples of the structural unit (a3) include non-acid-dissociable structural units derived from a monomer such as an aliphatic (meth)acrylate compound such as an alkyl (meth)acrylate, an alkoxyalkyl (meth)acrylate, or an alkoxy (poly)alkylene glycol (meth)acrylate, an alicyclic (meth)acrylate compound, an aromatic ring-containing (meth)acrylate compound, a styrene-based vinyl compound, an unsaturated nitrile compound, an unsaturated amide compound, or an unsaturated imide compound.

The polymer (A) can have one type or two or more types of structural units (a3).

When the polymer (A) has the structural unit (a3), the content ratio of the structural unit (a3) in the polymer (A) is usually 45 mol % or less.

When the polymer (A) has the structural unit (a3), the polymer (A) can have the structural unit (a3) in a polymer the same as or different from a polymer having the structural unit (a1) and/or (a2), and preferably has the structural units (a1) to (a3) in the same polymer.

### <Photoacid Generator (B)>

The photoacid generator (B) is a compound that generates an acid by exposure. By the action of this acid, the acid-dissociable group in the polymer (A) is dissociated, and an acidic functional group such as a carboxy group or a hydroxyaryl group is generated in the polymer (A).

As the photoacid generator (B), an onium salt compound, a halogen-containing compound, a sulfone compound, a sulfonimide compound, and a diazomethane compound can be used, and examples thereof include compounds described in [0017] to [0026], [0028] to [0039], [0042] to [0046], [0049], and [0053] in JP 2004-317907 A, compounds described in [0090] to [0106] in JP 2014-157252 A, compounds described in [0117] to [0123] in JP 2002-268223 A, and compounds described in [0038] to [0041] in JP 2017-102260 A.

The compounds described in the above-mentioned publications shall be described in the present description.

Examples of the photoacid generator (B) include onium salt compounds such as a compound represented by a formula (b1) or (b2) below, diphenyliodonium hexafluoroantimonate, diphenyliodonium hexafluorophosphate, diphenyliodonium tetrafluoroborate, triphenylsulfonium hexafluoroantimonate, triphenylsulfonium hexafluorophosphate, and phenylthiophenyl diphenylsulfonium tris(pentafluoroethyl)trifluorophosphate; halogen-containing compounds such as 1,10-dibromo-n-decane, 1,1-bis(4-chlorophenyl)-2,2,2-trichoroethane, phenyl-bis(trichloromethyl)-s-triazine, 4-methoxyphenyl-bis(tricholoromethyl)-s-triazine, styryl-bis(trichloromethyl)-s-triazine, and naphthyl-bis(trichloromethyl)-s-triazine; sulfone compounds such as 4-trisphenacylsulfone, mesitylphenacylsulfone, and bis(phenylsulfonyl)methane;
sulfonic acid compounds such as benzoin tosylate, pyrogallol tristrifluoromethanesulfonate, o-nitrobenzyl trifluoromethanesulfonate, and o-nitrobenzyl-p-toluenesulfonate; sulfonimide compounds such as N-(trifluoromethylsulfonyloxy)succinimide, N-(trifluoromethylsulfonyloxy)phthalimide, N-(trifluoromethylsulfonyloxy)diphenylmaleimide, N-(trifluoromethylsulfonyloxy)-4-butyl-naphthylimide, N-(4-methylphenylsulfonyloxy)succinimide, N-(4-methylphenylsulfonyloxy)phthalimide, N-(4-methylphenylsulfonyloxy)diphenylmaleimide, N-(4-methylphenylsulfonyloxy)bicyclo[2.2.1]hept-5-ene-2,3-dicarboximide, N-(4-methylphenylsulfonyloxy)bicyclo[2.1.1]heptan-5,6-oxy-2,3-dicarboximide, N-(4-fluorophenylsulfonyloxy)naphthylimide, and N-(10-camphor-sulfonyloxy)naphthylimide; and diazomethane compounds such as bis(trifluoromethylsulfonyl)diazomethane, bis(cyclohexylsulfonyl)diazomethane, bis(phenylsulfonyl)diazomethane, bis(p-toluenesulfonyl)diazomethane, methylsulfonyl-p-toluenesulfonyldiazomethane, cyclohexylsulfonyl-1,1-dimethylethylsulfonyldiazomethane, and bis(1,1-dimethylethylsulfonyl)diazomethane.

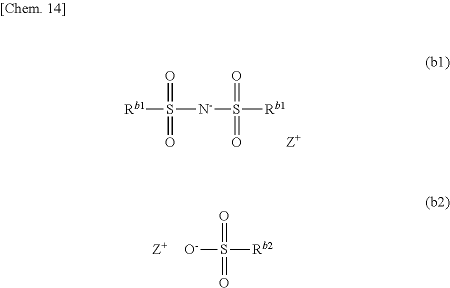

In the formulae (b1) and (b2), Z⁺ represents an onium cation, R^{b1} and R^{b2} each independently represent an alkyl group having 1 to 10 carbon atoms, a cycloalkyl group, an aryl group, or a group obtained by combining these groups, and the alkyl group, the cycloalkyl group, and the aryl group are unsubstituted or some or all hydrogen atoms in these groups are substituted with a fluorine atom, provided that a case where two R^{b1}'s represent a cyclic structure represented by bonding carbon atoms contained in these groups to each other is included.

Specific examples of the onium compound represented by the formulae (b1) and (b2) include onium salt compounds such as diphenyliodonium trifluoromethanesulfonate, diphenyliodonium p-toluenesulfonate, triphenylsulfonium trifluoromethanesulfonate, triphenylsulfonium nonafluorobutanesulfonate, triphenylsulfonium benzenesulfonate, 4-t-butylphenyl diphenylsulfonium trifluoromethanesulfonate, 4-t-butylphenyl diphenylsulfonium nonafluorobutanesulfonate, 4-t-butylphenyl diphenylsulfonium benzenesulfonate, 4,7-di-n-butoxynaphthyl tetrahydrothiophenium trifluoromethanesulfonate, 4,7-di-n-butoxynaphthyl tetrahydrothiophenium nonafluorobutanesulfonate, a 2,7-di-n-butoxynaphthyltetrahydrothiophenium bis(trifluoromethanesulfonyl)imide anion, a 2,7-di-n-butoxynaphthyltetrahydrothiophenium bis(nonafluorobutanesulfonyl)imide anion, a 4,7-di-n-butoxynaphthyltetrahydrothiophenium bis(trifluoromethanesulfonyl)imide anion, a 4,7-di-n-butoxynaphthyltetrahydrothiophenium bis(nonafluorobutanesulfonyl)imide anion, and a 4,7-di-n-butoxynaphthyltetrahydrothiophenium tris(nonafluorobutylsulfonyl)methide anion.

Among these compounds, an onium salt compound or a sulfonimide compound is preferable from the viewpoint of being able to form a resist pattern excellent in resolution and plating solution resistance, an onium salt compound is more preferable, and a compound represented by a formula (b1) or (b2) below is still more preferable among the onium salt compounds.

The present composition can contain one type or two or more types of photoacid generators (B).

The content ratio of the photoacid generator (B) in the present composition is 1 to 30 parts by mass, preferably 2 to 25 parts by mass, and more preferably 3 to 20 parts by mass with respect to 100 parts by mass of the polymer (A).

### <Solvent (C)>

The solvent (C) is an organic solvent. The organic solvent is, for example, a component used for uniformly mixing the respective components contained in the present composition.

Examples of the organic solvent include an alcohol solvent, an ester solvent, a ketone solvent, an alkylene glycol dialkyl ether, and an alkylene glycol monoalkyl ether acetate. Among them, an ester solvent, an alkylene glycol dialkyl ether, and an alkylene glycol monoalkyl ether acetate are preferable, and at least one selected from the group consisting of propylene glycol monomethyl ether acetate, 3-methoxybutyl acetate, and propylene glycol monomethyl ether is more preferable.

The present composition can contain one type or two or more types of solvents (C).

The content ratio of the solvent (C) in the present composition is usually 40 to 90 mass % in 100 mass % of the present composition. That is, the solid content concentration of the present composition is usually 10 to 60 mass %.

### <Compound (D)>

The compound (D) is a compound which is decomposed by the action of an acid generated from the photoacid generator (B) to become a compound (d), and is a compound having a group which is decomposed by the action of an acid to generate a compound (d′). However, the compound (D) is different from the polymer (A), and the compound (d) and the compound (d′) after decomposition are non-basic compounds at the time of pattern formation.

In addition, the compound (D) satisfies either the following requirement (D1) or (D2), and preferably satisfies both the following requirements (D1) and (D2).

### <<Requirement (D1)>>

The boiling point of the compound (d′) is 65° C. or higher, preferably 70° C. or higher, more preferably 80° C. or higher, still more preferably 90° C. or higher, and particularly preferably 100° C. or higher. The upper limit of the boiling point is not particularly limited, but is preferably 300° C. or lower.

When the boiling point of the compound (d′) is in the above range, unevenness (wave-shape) of the pattern shape due to a standing wave tends to be improved.

### <<Requirement (D2)>>

(D2): The compound (D) has a group represented by a formula (i) below.

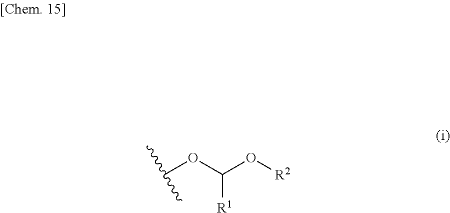

In the formula (i), R¹ represents an alkyl group having 1 to 3 carbon atoms, R² represents an alkyl group having 2 to 10 carbon atoms, a cycloalkyl group having 3 to 20 carbon atoms, an aryl group having 6 to 10 carbon atoms, a group obtained by combining these groups, or a group in which these groups are linked via an ether bond, or represents a cyclic structure represented by bonding carbon atoms contained in these groups of R¹ and R² to each other. That is, R¹ and R² may be bonded to each other to form a cyclic structure. R² is preferably an alkyl group having 2 to 10 carbon atoms or a cycloalkyl group having 3 to 20 carbon atoms.

Examples of the alkyl groups having 1 to 3 carbon atoms and 2 to 10 carbon atoms include those having a similar carbon number as the corresponding carbon number among those exemplified for the alkyl group having 1 to 10 carbon atoms, examples of the cycloalkyl group having 3 to 20 carbon atoms include those having a similar carbon number as the corresponding carbon number among those exemplified for the cycloalkyl group of R^{a1} to R^{a3}, and examples of the aryl group having 6 to 10 carbon atoms include a phenyl group and a naphthyl group.

The group represented by the formula (i) is the “group which is decomposed by the action of an acid to generate the compound (d′)”, and after the decomposition, R²OH is generated as the compound (d′). As described above, the compound (D) preferably satisfies both the requirements (D1) and (D2), that is, the boiling point of R²OH generated as the compound (d′) is preferably 65° C. or higher.

Preferred specific examples of the group represented by the formula (i) include groups represented by formulae (i-1) to (i-3) below.

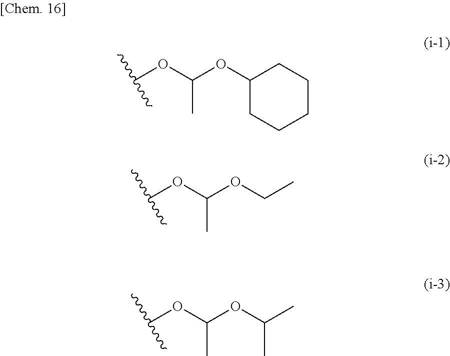

In the case of the group represented by the formula (i-1), the compound (d′) after decomposition has a boiling point of 162° C. (cyclohexanol; catalogue value), in the case of the group represented by the formula (i-2), the compound (d′) after decomposition has a boiling point of 78° C. (ethanol; catalog value), and in the case of the group represented by the formula (i-3), the compound (d′) after decomposition has a boiling point of 83° C. (iso-propanol; catalog value).

In addition, the compound (D) is preferably a compound represented by any one of formulae (I) to (IV) below.

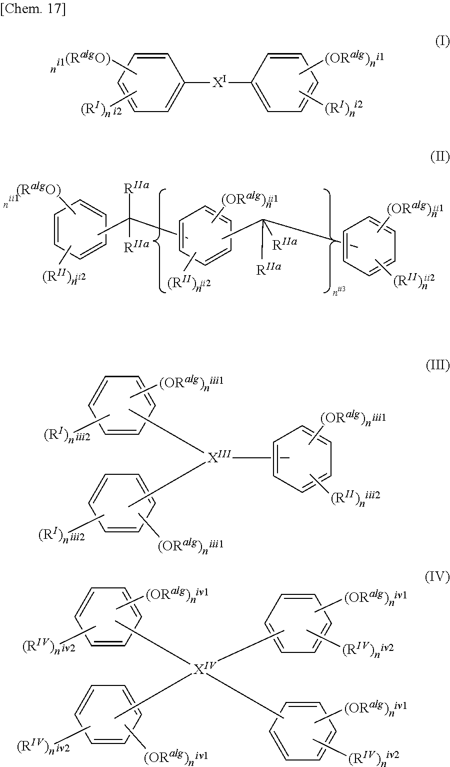

In the formulae (I) to (IV), R^{alg}'s independently represent a hydrogen atom or a group that generates the compound (d′), R^{I} to R^{IV} each independently represent an alkyl group having 1 to 4 carbon atoms, a cycloalkyl group having 3 to 10 carbon atoms, an aryl group having 6 to 10 carbon atoms, or a halogen atom, nⁱ¹ to n^{iv1} and nⁱⁱ³ each independently represent an integer of 1 or 2, and nⁱ² to n^{iv2} each independently represent an integer of 0 to 2.

In the formula (II), R^{IIa}'s independently represent a hydrogen atom or an alkyl group having 1 or 2 carbon atoms.

In the formula (I), X^{I} represents a single bond or a structure represented by formulae (I-1) to (I-3) below.

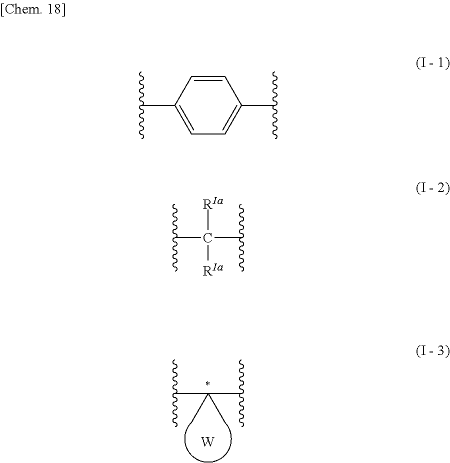

In the formula (I-2), R^{Ia} represents a hydrogen atom, an alkyl group having 1 to 10 carbon atoms, a cycloalkyl group having 3 to 10 carbon atoms, an aryl group having 6 to 10 carbon atoms, a halogen atom, or a group represented by a formula (I-2a) below. In the formula (I-2a) below, R^{alg}, R^{Ia}, nⁱ¹, and nⁱ² indicate similar meanings to the same symbols in the formula (I).

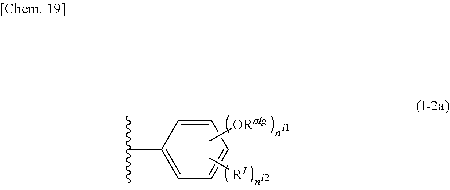

In the formula (I-3), W represents a structure represented by a formula (I-3a) or (I-3b) below. In the formulae (I-3a) and (I-3b) below, a carbon atom with * indicates the same carbon atom as * in the formula (I-3)

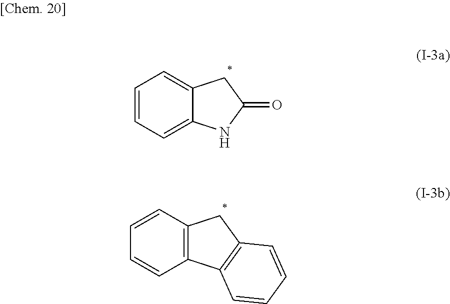

In the formula (III), X^{III} represents a structure represented by a formula (III-1) or (III-2) below: In the formulae (III-1) and (III-2) below, R^{IIIa} represents a substituent similar to R^{IIa} in the formula (II).

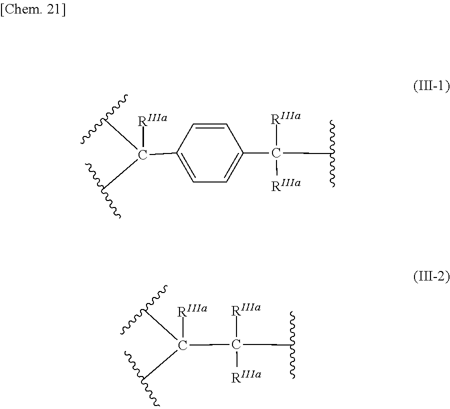

In the formula (IV), X^{IV} represents a structure represented by any of formulae (IV-1) to (IV-3) below. In the formulae (IV-1) to (IV-3) below, R^{IVa} represents a substituent similar to R^{IIa} in the formula (II).

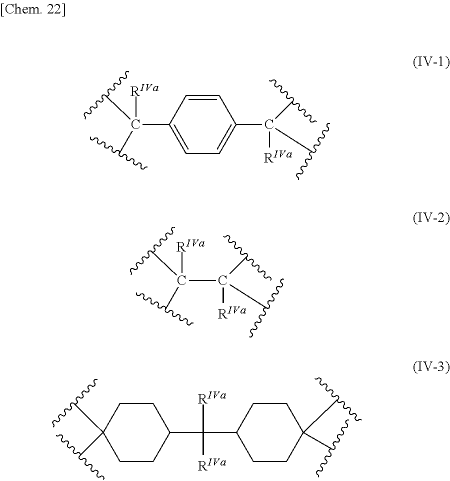

The compound (D) tends to have an excellent balance between a standing wave and trailing, and thus is preferably a compound represented by the formula (I) or (IV), and more preferably a compound represented by the formula (I) in which X^{I} in the formula (I) has a structure represented by the formula (I-2) or a compound represented by the formula (IV) in which X^{IV} in the formula (IV) has a structure represented by the formula (IV-2).

In addition, the compound (D) contained in the present composition preferably has two or more groups that generate the compound (d′) on average in one molecule. The number of groups that generate the compound (d′) in one molecule of the compound (D) is not particularly limited, but is preferably 8 or less on average. When the number of groups is in the above range, the balance between a standing wave and trailing occurring in a resist pattern film to be obtained tends to be excellent.

As the compound (D), compounds represented by formulae (D1) to (D6) below are preferable. In addition, the compounds represented by the formulae (D2) to (D4) below are a mixture of compounds in which R represents a group represented by the formula (i-1) or a hydroxy group, and the formulae (D2) to (D4) below have 1 or more groups represented by the formula (i-1) on average in one molecule.

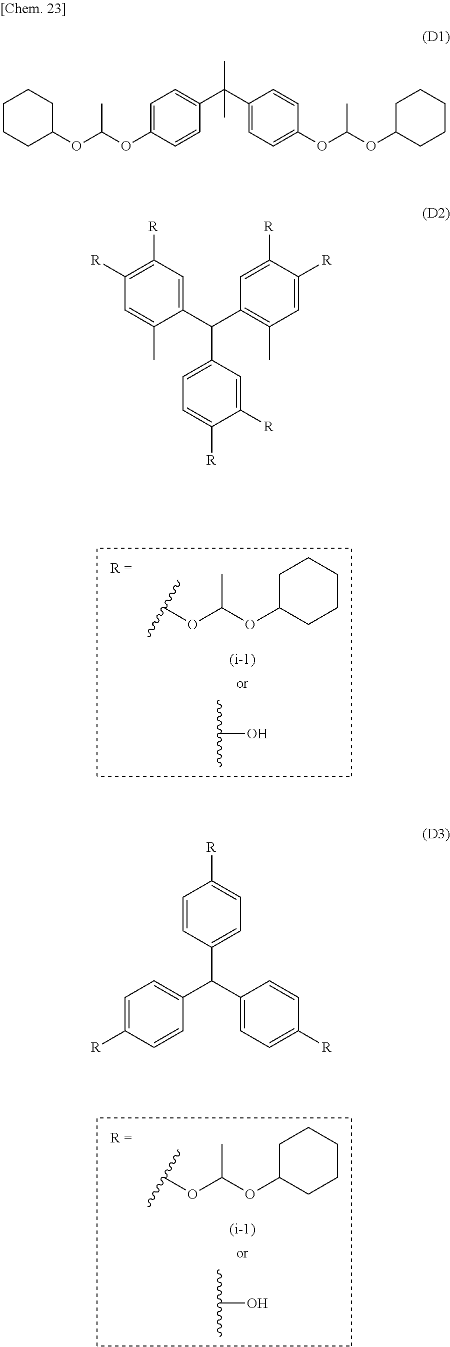

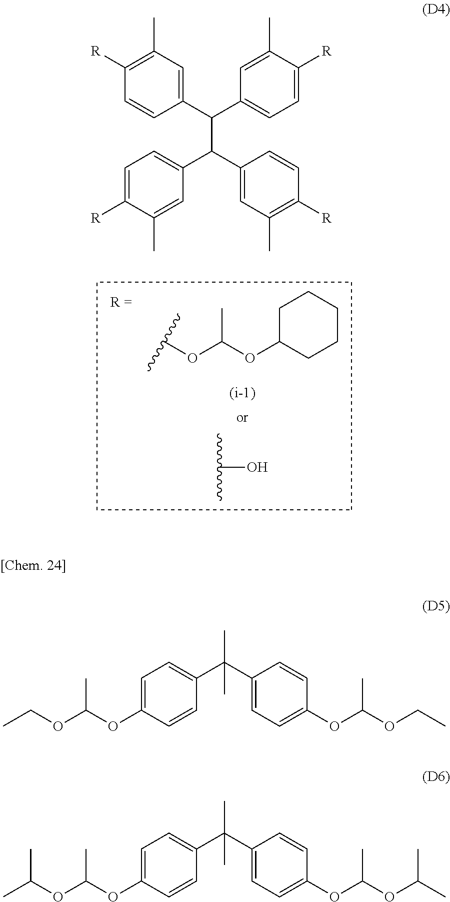

The present composition can contain one type or two or more types of compounds (D).

The content ratio of the compound (D) in the present composition is 1 to 40 parts by mass, preferably 2 to 30 parts by mass, and more preferably 3 to 20 parts by mass with respect to 100 parts by mass of the polymer (A). When the content ratio of the compound (D) is in the above range, the balance between a standing wave and trailing occurring in a resist pattern film to be obtained tends to be excellent.

### <Quencher (E)>

The present composition may further contain a quencher (E). The quencher (E) is used for controlling diffusion of an acid generated by exposure from the photoacid generator (B) in the resin film, and, as a result, can improve the resolution of the present composition. The quencher (E) is preferably a compound (E1) (hereinafter also referred to as “compound (E1)”) which is decomposed by the action of an acid to generate a primary or secondary amine from the viewpoint of improving resolution.

Examples of the compound (E1) include nitrogen-containing compounds having an acid-dissociable group such as N-(t-butoxycarbonyl)piperidine, N-(t-butoxycarbonyl)-4-hydroxypiperidine, N-(t-butoxycarbonyl)imidazole, N-(t-butoxycarbonyl)benzimidazole, N-(t-butoxycarbonyl)-2-phenylbenzimidazole, and N-(t-amyloxycarbonyl)-2-phenylbenzimidazole.

Examples of another quencher (E) other than the compound (E1) include trioctylamine and triethanolamine.

The present composition can contain one type or two or more types of compounds (E).

The content of the compound (E) in the present composition is usually 1 to 20 mol %, and preferably 2 to 18 mol % with respect to 100 mol % of the photoacid generator (B).

### <Other Components>

The present composition can further contain other components other than the components (A) to (E) as long as the effects of the present invention are not impaired. Examples of the other components include a surfactant that exhibits an action of improving, for example, coating applicability and defoamability of the present composition, a sensitizer that absorbs exposure light to improve the acid generation efficiency of a photoacid generator, an alkali-soluble resin (G) or a low molecular weight phenol compound that controls a dissolution rate of a resin film formed from the present composition in an alkaline developer, an ultraviolet absorber that blocks a photoreaction due to wraparound of scattered light to an unexposed portion during exposure, a thermal polymerization inhibitor that enhances the storage stability of the positive composition, an antioxidant, an adhesion aid, and an inorganic filler.

In addition to the above, when the present composition is used as a template for a plated formed product, a thiol group-containing compound (F) can be added as an additive that acts on a Cu surface to prevent a quenching action of the Cu surface so as to be able to improve the trailing shape of the resist bottom.

### <Method for Producing Photosensitive Resin Composition>

The present composition can be produced by uniformly mixing the respective components described above. In addition, in order to remove foreign substances, after the above-described respective components are uniformly mixed, the obtained mixture can be filtered with a filter such as a membrane filter or a capsule cartridge filter.

### [Method for Producing Resist Pattern Film]

A method for producing a resist pattern film according to an aspect of the present invention (hereinafter also referred to as “present method for producing a resist pattern film”) includes a step (1) of forming a resin film of a photosensitive resin composition containing a polymer (A) having an acid-dissociable group, a photoacid generator (B), a solvent (C), and a compound (D) on a metal film of a substrate having the metal film, a step (2) of exposing the resin film to light, and a step (3) of developing the resin film after exposure to form a resist pattern film.

### <Step (1)>

In the step (1), a resin film of a photosensitive resin composition containing a polymer (A) having an acid-dissociable group, a photoacid generator (B), a solvent (C), and a compound (D) is formed on a metal film of a substrate having the metal film.

Examples of the substrate include a semiconductor substrate and a glass substrate. The shape of the substrate is not particularly limited, and examples of the surface shape include a flat plate shape and an uneven shape, and examples of the shape of the substrate include a circular shape and a square shape. The size of the substrate is not limited.

Examples of the metal film include films containing a metal such as aluminum, copper, silver, gold, or palladium, or two or more alloys containing the metal, and a copper film, that is, a film containing copper and/or a copper alloy is preferable. The thickness of the metal film is usually 100 to 10,000 Å, and preferably 500 to 2,000 Å. The metal film is usually provided on the surface of the substrate. The metal film can be formed by a method such as a sputtering method.

The resin film is usually formed by applying the composition onto a metal film of a substrate having the metal film. Examples of the method for applying the composition include a spin coating method, a roll coating method, a screen printing method, and an applicator method, and among these methods, a spin coating method and a screen printing method are preferable.

After applying the composition, a heat treatment can be performed for the applied composition for the purpose of volatilizing the solvent, for example. The conditions of the heat treatment are usually 50 to 200° C. for 0.5 to 20 minutes. The thickness of the resin film is preferably 0.5 to 100 μm, and more preferably 5 to 80 μm.

### <Step (2)>

In the step (2), the resin film formed in the step (1) is exposed to light. The exposure is usually selectively performed on the resin film by equal magnification projection exposure or reduction projection exposure via a photomask having a predetermined mask pattern. Examples of the exposure light include ultraviolet light or visible light having a wavelength of 150 to 600 nm, preferably 200 to 500 nm. Examples of the light source of the exposure light include a low-pressure mercury lamp, a high-pressure mercury lamp, an ultra-high-pressure mercury lamp, a metal halide lamp, and a laser. The exposure amount can be appropriately selected depending on the type of exposure light, the type of photosensitive resin composition, and the thickness of the resin film, and is usually 100 to 20,000 mJ/cm².

After exposure of the resin film, a heat treatment can be performed for the resin film before development. The conditions of the heat treatment are usually 70 to 180° C. for 0.5 to 10 minutes. By the heat treatment, the dissociation reaction of the acid-dissociable group by the acid in the polymer (A) can be promoted.

### <Step (3)>

In the step (3), the resin film exposed in the step (2) is developed to form a resist pattern film. The development is usually performed using an alkaline developer. Examples of the developing method include a shower method, a spray method, an immersion method, a liquid filling method, and a paddle method. The development conditions are usually 10 to 30° C. for 1 to 30 minutes.

Examples of the alkaline developer include an aqueous solution containing one type or two or more types of alkaline substances. Examples of the alkaline substance include sodium hydroxide, potassium hydroxide, sodium carbonate, sodium silicate, ammonia water, ethylamine, n-propylamine, diethylamine, triethylamine, monoethanolamine, diethanolamine, triethanolamine, tetramethylammonium hydroxide, tetraethylammonium hydroxide, choline, pyrrole, and piperidine. The concentration of the alkaline substance in the alkaline developer is usually 0.1 to 10 mass %. The alkaline developer can further contain, for example, an organic solvent such as methanol or ethanol and/or a surfactant.

The resist pattern film formed by development can be washed with water, for example. Thereafter, the resist pattern film can be dried using an air gun or a hot plate.

As described above, it is possible to form a resist pattern film with a rectangular cross section to serve as a mold for forming a plated formed product on a metal film of a substrate, and a plating substrate having a resist pattern film (mold) on a metal film is obtained.

The thickness of the resist pattern film is preferably 0.5 to 100 μm, and more preferably 5 to 80 μm. Usually, when the thickness of the resist pattern film is 0.5 to 100 μm, a resist pattern film with a non-rectangular cross section such as a footing-shaped cross section is easily formed. Therefore, when the thickness of the resist pattern film is 0.5 to 100 μm, the effect of the present method for producing a present resist pattern film becomes more apparent.

As the shape of the opening of the resist pattern film, a shape suitable for the type of plated formed product can be selected. When the plated formed product is a wiring line, the shape viewed from above the opening of the resist pattern film is linear, and when the plated formed product is a bump, the shape viewed from above the opening of the resist pattern film is square.

The width of the resist pattern film is usually 1 to 300 μm, and preferably 15 to 100 μm. The ratio (T/W) of the width (W) of the resist pattern film to the thickness (T) of the resist pattern film is usually 0.5 or more, and preferably 1 to 10. Within the above range, the effect of the method for producing a resist pattern film of the present invention becomes more apparent.

### [Method for Producing Plated Formed Product]

A method for producing a plated formed product according to an aspect of the present invention (hereinafter also referred to as “present method for producing a plated formed product”) includes a step (4) of performing a plating treatment using a resist pattern film formed by the present method for producing a resist pattern film as a template.

### <Step (4)>

The plating treatment may be performed by a known method, and examples thereof include wet plating treatments such as an electroplating treatment, an electroless plating treatment, and a hot dipping treatment, and dry plating treatments such as chemical vapor deposition and sputtering.

In the case of forming a wiring line or a connection terminal in processing at a wafer level, the formation is usually performed by an electroplating treatment.

Before the plating treatment is performed, a pre-treatment such as an ashing treatment, a flux treatment, or a desmear treatment can be performed in order to enhance the affinity between the surface of the inner wall of the resist pattern film and the plating solution.

In a case where the plating treatment is an electroplating treatment, one formed on the inner wall of the resist pattern film by sputtering or an electroless plating treatment can be used as a seed layer, and in the case of using, as the substrate, a substrate having a metal film on a surface, the metal film can also be used as a seed layer. A barrier layer may be formed before the seed layer is formed, or the seed layer can also be used as a barrier layer.

Examples of the plating solution used for an electroplating treatment include a copper plating solution containing, for example, copper sulfate or copper pyrophosphate; a gold plating solution containing gold potassium cyanide; a nickel plating solution containing nickel sulfate or nickel carbonate; and a tin plating solution containing a tin alkyl sulfonate.

In the present method for producing a plated formed product, the plating treatment is preferably one selected from copper, nickel, tin, and gold plating treatments using, for example, the plating solution.

The conditions for the electroplating treatment can be appropriately selected depending on, for example, the type of plating solution, and for example, in the case of an electroplating treatment containing copper sulfate, the temperature is usually 10 to 90° C. and the current density is 0.1 to 100 A/dm². In the plating treatment, different plating treatments can be sequentially performed. For example, a solder copper pillar bump can be formed by first performing a copper plating treatment, subsequently performing a nickel plating treatment, and then performing a molten solder dipping treatment.

The thickness of the plated formed product varies depending on the application, but for example, when the plated formed product is a bump, the thickness is usually 5 to 200 μm, and when the plated formed product is a wiring line, the thickness is usually 1 to 30 μm.

### <Other Steps>

In the present method for producing a plated formed product, examples of other steps other than the steps (1) to (4) include a step of removing the resist pattern film (hereinafter also referred to as “step (5)”) after the step (4). The step (5) is performed with, for example, a resist stripping liquid containing tetramethylammonium hydroxide, dimethyl sulfoxide, and/or N,N-dimethylformamide. The present method for producing a plated formed product may further include a step of removing the metal film in a region other than the region where the plated formed product is formed, for example, by a wet etching method.

### EXAMPLES

Hereinafter, the present invention will be described more specifically based on examples, but the present invention is not limited to these examples.

### <Measurement of Weight Average Molecular Weight (Mw) of Polymer>

The weight average molecular weight (Mw) of a polymer was measured by gel permeation chromatography (GPC) under the following conditions.
GPC apparatus: apparatus name “HLC-8220-GPC” manufactured by TOSOH CORPORATION
Column: Columns “TSK-M” and “TSK 2500” manufactured by TOSOH CORPORATION connected in series, solvent: tetrahydrofuran
Temperature: 40° C.
Detection method: refractive index method
Standard substance: polystyrene

The details of respective components used in the following examples and comparative examples are as follows.

### <Polymer (A)>

(A1): a polymer having a structural unit described in (A1) below (the numerical value described in each structural unit represents a molar ratio (mol %); Mw=38,600)
(A2): a polymer having a structural unit described in (A2) below (the numerical value described in each structural unit represents a molar ratio (mol %); Mw=40,000)
(A3): a polymer having a structural unit described in (A3) below (the numerical value described in each structural unit represents a molar ratio (mol %); Mw=39,000)
(A4): a polymer having a structural unit described in (A4) below (the numerical value described in each structural unit represents a molar ratio (mol %); Mw=43,000)
(A5): a polymer having a structural unit described in (A5) below (the numerical value described in each structural unit represents a molar ratio (mol %); Mw=48,000)

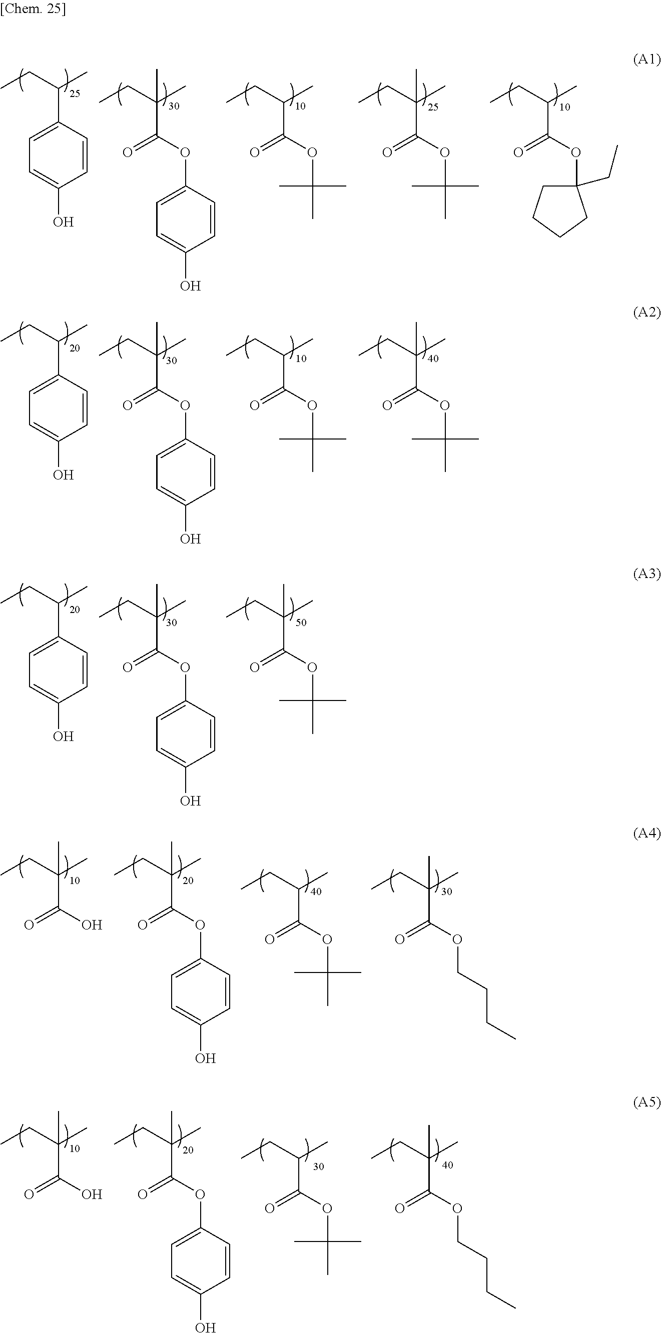

### <Photoacid Generator (B)>

(B1): a compound represented by a formula (B1) below
(B2): a compound represented hv a formula (B2) below
(B3): a compound represented by a formula (B3) below

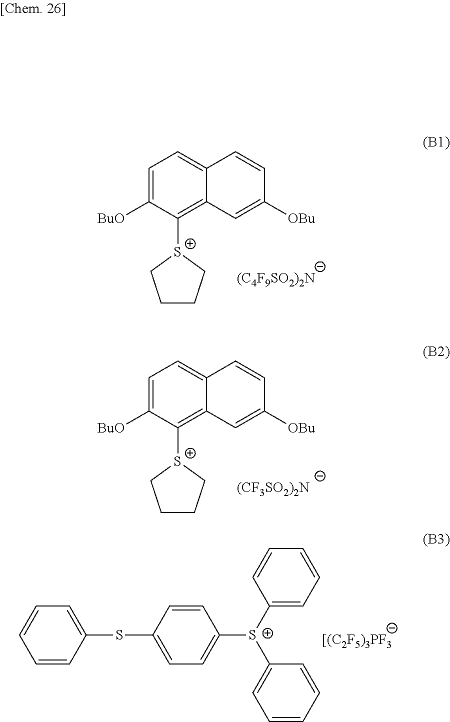

### <Solvent (C)>

(C1): propylene glycol monomethyl ether acetate (PGMEA)
(C2): propylene glycol monomethyl ether (PGME)

### <Compound (D)>

(D1): a compound represented by a formula (D1) below
(D2): a compound represented by a formula (D2) below (having 5 groups represented by the formula (i-1) on average in one molecule)
(D3): a compound represented by a formula (D3) below (having 3 groups represented by the formula (i-1) on average in one molecule)
(D4): a compound represented by a formula (D4) below (having 4 groups represented by the formula (i-1) on average in one molecule)
(D5): a compound represented by a formula (D5) below
(D6): a compound represented by a formula (D6) below
(d1): a compound represented by a formula (d1) below (which is decomposed by the action of an acid to generate isobutene (boiling point: −7° C.))

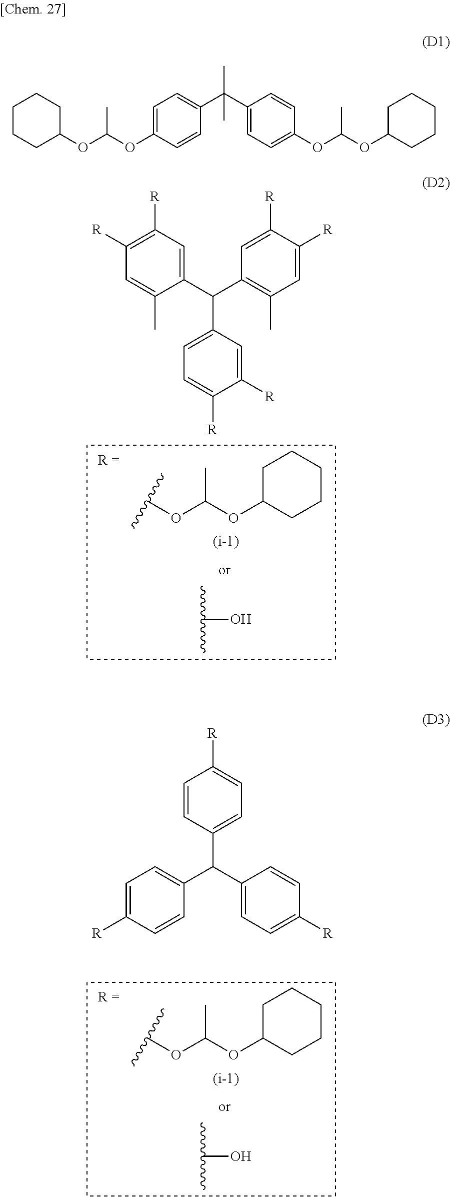

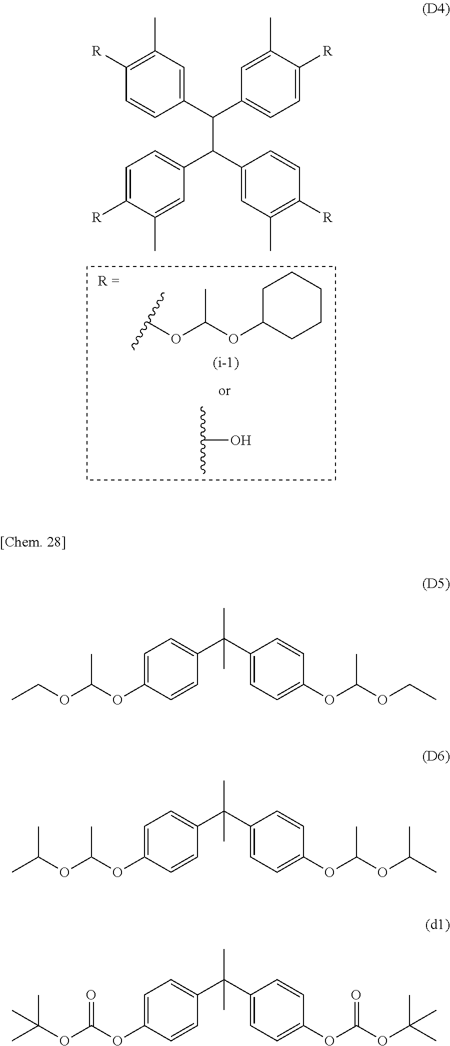

### <Quencher (E)>

(E1-1): a compound represented by a formula (E1-1) below
(E1-2): a compound represented by a formula (E1-2) below
(E2): trioctylamine

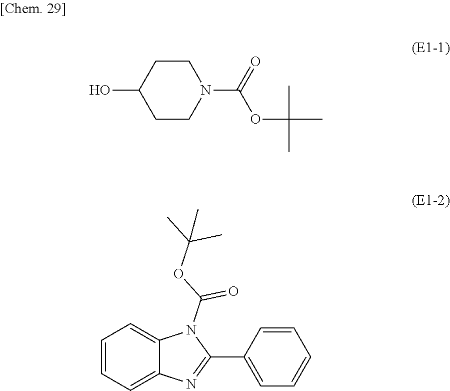 [SMILES: CC(C)(C)OC(=O)N1CCC(O)CC1 ; CC(C)(C)OC(=O)n1c(-c2ccccc2)nc2ccccc21]

### <Other Components>

### Thiol Group-Containing Compound (F)

(F1): a compound represented by a formula (F1) below
(F2): a compound represented by a formula (F2) below
(F3): a compound represented by a formula (F3) below

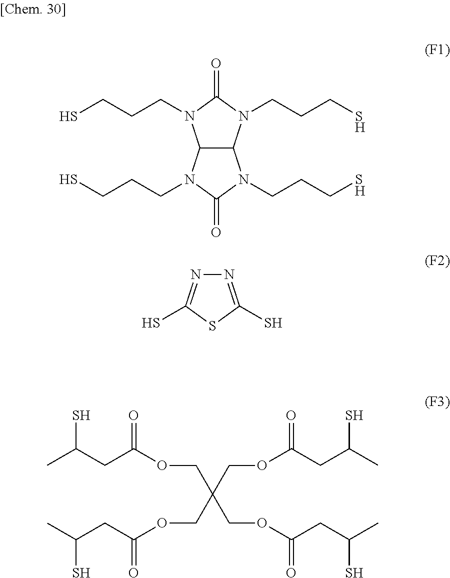

### Alkali-Soluble Resin (G)

(G1): a polymer having a structural unit described in (G1) below (the numerical value described in each structural unit represents a molar ratio (mol %); Mw=9,850)

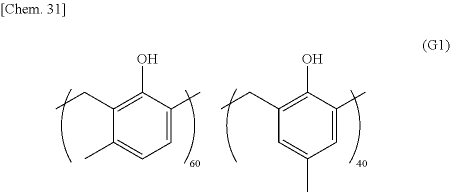

### Example 1

A photosensitive resin composition was produced by uniformly mixing 100 parts by mass of the polymer (A1), 6 parts by mass of the photoacid generator (B1), 15 parts by mass of the compound (D1), and the quencher (E1-1) in an amount of 15 mol % with respect to 100 mol % of the photoacid generator (B1) with the solvent (C1) so as to have a solid content concentration of 18 mass %.

### Examples 2 to 21, Comparative Examples 1 to 4

Photosensitive resin compositions were produced in the same manner as in Example 1 except that the respective components in Example 1 were changed to components and amounts shown in Table 1. The amount (mol %) of the quencher (E) shown in Table 1 indicates the amount (mol %) with respect to 100 mol % of the photoacid generator (B).

### <Evaluation (Standing Wave, Trailing)>

The photosensitive resin composition obtained in each of examples and comparative examples was applied onto a copper sputtered film of a silicon wafer substrate provided with the copper sputtered film using a spin coater, and heated at 120° C. for 60 seconds to form a coating film having a film thickness of 1.5 μm. The coating film was exposed via a pattern mask using a stepper (model “NSR-i12D”, manufactured by Nikon Corporation). The coating film after exposure was heated at 90° C. for 60 seconds, and then developed by immersion in a 2.38 mass % aqueous tetramethylammonium hydroxide solution for 80 seconds. Thereafter, the substrate was washed with flowing water and blown with nitrogen to form a resist pattern film (line width: 0.5 μm, line width/space width=1/1) on the copper sputtered film of the substrate. The substrate on which the resist pattern film was formed is referred to as a patterning substrate.

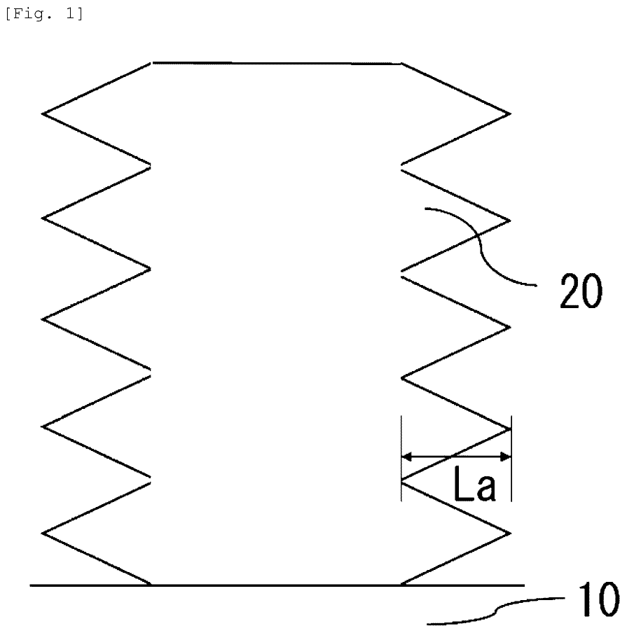

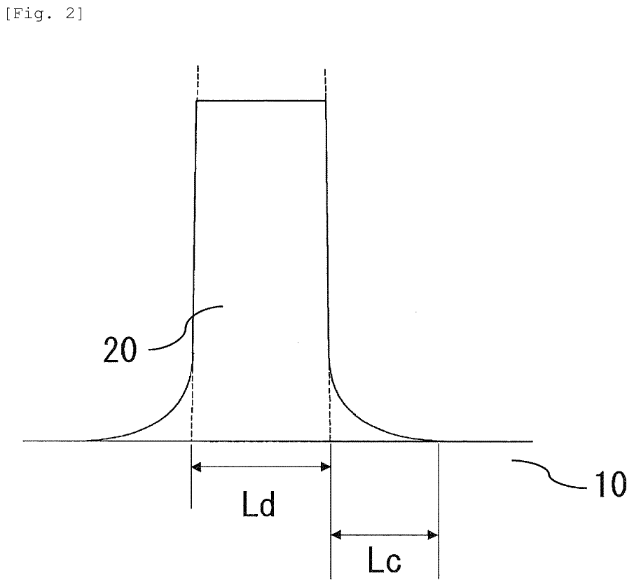

In the obtained patterning substrate, the state of the interface between the resist pattern film and the copper sputtered film was observed. The obtained cross section of 1L (line) 1S (space) having a line width of 0.5 μm was observed using a scanning electron microscope, and a width La (line width at a place where the unevenness (wave-shape) of a generated standing wave is most significant) illustrated in FIG. 1 and a width Lc and a width Ld illustrated in FIG. 2 were measured and evaluated according to the following criteria.

In FIGS. 1 and 2, the unevenness (wave-shape) generated in the pattern and the trail of the pattern are exaggerated from the actual state. The evaluation results are shown in Table 1.

### (Standing Wave)

A: La is less than 10 nm.
B: La is 10 nm or more and 20 nm or less.
C: La is more than 20 nm.

### (Trailing)

A: Lc/Ld is less than 0.05.
B: Lc/Ld is 0.05 or more and less than 0.2.
C: Lc/Ld is 0.2 or more.

TABLE 1

| | Formulation | | | | | | | | | | | | | | |
|---|---|---|---|---|---|---|---|---|---|---|---|---|---|---|---|
| | | | | | | Photoacid | | | | | | | | | |
| | | | | | | generator | | | | | | | | | |
| | Polymer (A) | | | | | (B) (parts | | | | | | | | | |
| | (parts by mass) | | | | | by mass) | | | Compound (D) (parts by mass) | | | | | | |
| | A1 | A2 | A3 | A4 | A5 | B1 | B2 | B3 | D1 | D2 | D3 | D4 | D5 | D6 | d1 |
| Example 1 | 100 | | | | | 6 | | | 15 | | | | | | |
| Example 2 | 100 | | | | | 6 | | | | 6 | | | | | |
| Example 3 | 100 | | | | | 6 | | | | | 12 | | | | |
| Example 4 | 100 | | | | | 6 | | | | | | 7 | | | |
| Example 5 | 100 | | | | | 6 | | | | | | | 12 | | |
| Example 6 | 100 | | | | | 6 | | | | | | | | 15 | |
| Example 7 | 100 | | | | | 6 | | | | | 7 | | | | |
| Example 8 | 100 | | | | | 6 | | | | | 17 | | | | |
| Example 9 | | 100 | | | | 6 | | | 15 | | | | | | |
| Example 10 | | | 100 | | | 6 | | | 15 | | | | | | |
| Example 11 | 100 | | | | | | 6 | | 15 | | | | | | |
| Example 12 | 100 | | | | | | | 5 | 15 | | | | | | |
| Example 13 | 100 | | | | | 6 | | | 15 | | | | | | |
| Example 14 | 100 | | | | | 6 | | | 15 | | | | | | |
| Example 15 | 100 | | | | | 6 | | | 15 | | | | | | |
| Example 16 | 100 | | | | | 6 | | | | | | 7 | | | |
| Example 17 | 100 | | | | | 6 | | | | | | 7 | | | |
| Example 18 | 100 | | | | | 6 | | | | | | 7 | | | |
| Example 19 | 80 | | | | | 6 | | | 15 | | | | | | |
| Example 20 | | | | 100 | | 6 | | | 15 | | | | | | |
| Example 21 | | | | | 100 | 6 | | | 15 | | | | | | |
| Comparative | 100 | | | | | 6 | | | | | | | | | 13 |
| Example 1 | | | | | | | | | | | | | | | |
| Comparative | 100 | | | | | | 6 | | | | | | | | 13 |
| Example 2 | | | | | | | | | | | | | | | |
| Comparative | 100 | | | | | 6 | | | | | | | | | 5 |
| Example 3 | | | | | | | | | | | | | | | |
| Comparative | 100 | | | | | 6 | | | | | | | | | |
| Example 4 | | | | | | | | | | | | | | | |

| | Formulation | | | | | | | | | |
|---|---|---|---|---|---|---|---|---|---|---|
| | Quencher (E) | | | Other components | | | | | Evaluation | |
| | (mol %) | | | (parts by mass) | | | | Solvent | Standing | |
| | E1-1 | E1-2 | E2 | F1 | F2 | F3 | G1 | (C) | wave | Trailing |
| Example 1 | 15 | | | | | | | C1 | A | B |
| Example 2 | 15 | | | | | | | C1 | A | A |
| Example 3 | 15 | | | | | | | C1 | A | B |
| Example 4 | 15 | | | | | | | C1 | A | A |
| Example 5 | 15 | | | | | | | C1 | B | B |
| Example 6 | 15 | | | | | | | C1 | B | B |
| Example 7 | 15 | | | | | | | C1 | B | A |
| Example 8 | 15 | | | | | | | C1 | A | B |
| Example 9 | 15 | | | | | | | C1 | A | B |
| Example 10 | 15 | | | | | | | C1 | A | B |
| Example 11 | 15 | | | | | | | C1 | A | B |
| Example 12 | 5 | | | | | | | C1 | B | B |
| Example 13 | 15 | | | | | | | C2 | A | B |
| Example 14 | | 15 | | | | | | C1 | B | B |
| Example 15 | | | 15 | | | | | C1 | B | B |
| Example 16 | 15 | | | 0.1 | | | | C1 | A | A |
| Example 17 | 15 | | | | 0.1 | | | C1 | A | B |
| Example 18 | 15 | | | | | 0.1 | | C1 | A | B |
| Example 19 | 15 | | | | | | 20 | C1 | A | B |
| Example 20 | 15 | | | | | | | C1 | A | B |
| Example 21 | 15 | | | | | | | C1 | A | B |
| Comparative | 15 | | | | | | | C1 | C | A |
| Example 1 | | | | | | | | | | |
| Comparative | 15 | | | | | | | C1 | C | A |
| Example 2 | | | | | | | | | | |
| Comparative | 15 | | | | | | | C1 | C | A |
| Example 3 | | | | | | | | | | |
| Comparative | 15 | | | | | | | C1 | C | A |
| Example 4 | | | | | | | | | | |

### REFERENCE SIGNS LIST

10 Substrate having metal film
20 Resist pattern film

1: A photosensitive resin composition comprising a polymer (A) which comprises an acid-dissociable group, a photoacid generator (B), a solvent (C), and a compound (D), wherein
the compound (D) is a compound which is decomposed by an action of an acid generated from the photoacid generator (B) to become a compound (d) and which has a group which is decomposed by an action of an acid to generate a compound (d′), provided that the compound (D) is different from the polymer (A), and the compound (d) and the compound (d′) are non-basic compounds, and
the compound (d′) has a boiling point of 65° C. or higher.

2: The photosensitive resin composition according to claim 1, wherein the compound (D) has a group represented by formula (i): 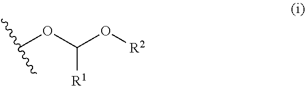
wherein, in the formula (i), R¹ represents an alkyl group having 1 to 3 carbon atoms, R² represents an alkyl group having 2 to 10 carbon atoms, a cycloalkyl group having 3 to 20 carbon atoms, an aryl group having 6 to 10 carbon atoms, a group obtained by combining at least two of the alkyl group, the cycloalkyl group, and the aryl group, or a group in which at least two of the alkyl group, the cycloalkyl group, and the aryl group are linked via an ether bond, or represents a cyclic structure represented by bonding carbon atoms contained in the groups of R¹ and R² to each other.

3: A photosensitive resin composition comprising a polymer (A) which comprises an acid-dissociable group, a photoacid generator (B), a solvent (C), and a compound (D),
the compound (D) is a compound which is decomposed by an action of an acid generated from the photoacid generator (B) to become a compound (d) and which is decomposed by an action of an acid to generate a compound (d′), provided that the compound (D) is different from the polymer (A), and the compound (d) and the compound (d′) are non-basic compounds, and
the compound (D) has a group represented by formula (i): 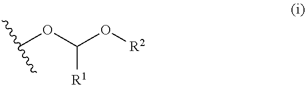
wherein, in the formula (i), R¹ represents an alkyl group having 1 to 3 carbon atoms, R² represents an alkyl group having 2 to 10 carbon atoms, a cycloalkyl group having 3 to 20 carbon atoms, an aryl group having 6 to 10 carbon atoms, a group obtained by combining at least two of the alkyl group, the cycloalkyl group, and the aryl group, or a group in which at least two of the alkyl group, the cycloalkyl group, and the aryl group are linked via an ether bond, or represents a cyclic structure represented by bonding carbon atoms contained in the groups of R¹ and R² to each other.

4: The photosensitive resin composition according to claim 1, wherein the compound (D) is represented by any one of formulae (I) to (IV): 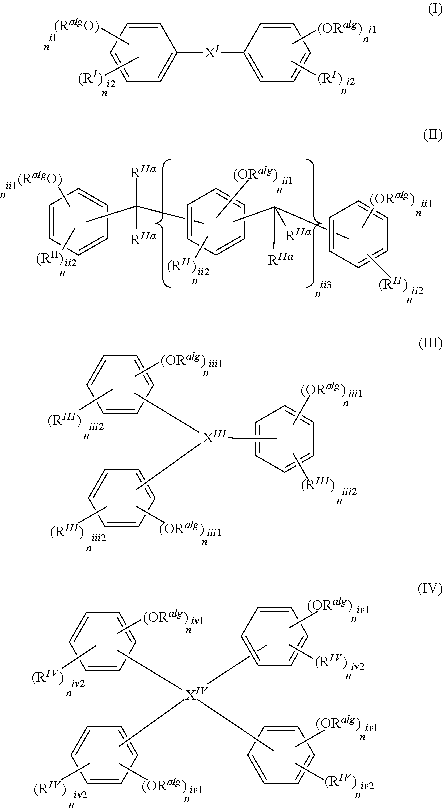
wherein, in the formulae (I) to (IV),
R^{alg}'s each independently represent a hydrogen atom or a group that generates the compound (d′);
R^{I} to R^{IV} each independently represent an alkyl group having 1 to 4 carbon atoms, a cycloalkyl group having 3 to 10 carbon atoms, an aryl group having 6 to 10 carbon atoms, or a halogen atom;
R^{IIa}'s each independently represent a hydrogen atom or an alkyl group having 1 or 2 carbon atoms;
nⁱ¹ to n^{iv1} and nⁱⁱ³ each independently represent an integer of 1 or 2;
nⁱ² to n^{iv2} each independently represent an integer of 0 to 2;
X¹ represents a single bond or a structure represented by formulae (I-1) to (I-3): 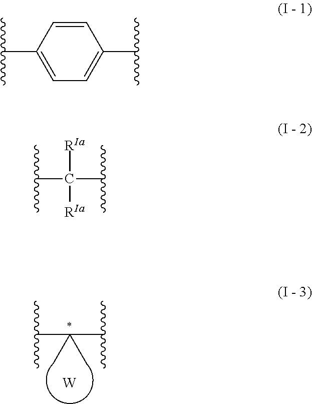
wherein, in the formula (I-2), R^{Ia} represents a hydrogen atom, an alkyl group having 1 to 10 carbon atoms, a cycloalkyl group having 3 to 10 carbon atoms, an aryl group having 6 to 10 carbon atoms, a halogen atom, or a group represented by formula (I-2a): 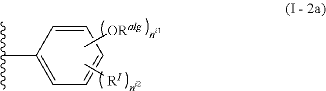
wherein, R^{alg}, R^{I}, nⁱ¹ and nⁱ² are each as defined in the formula (I),
in the formula (I-3), W represents a structure represented by formula (I-3a) or (I-3b): 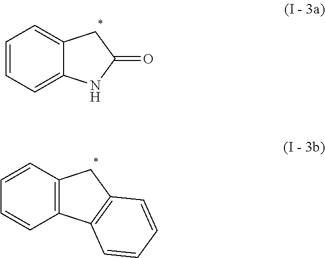
wherein, in the formulae (I-3a) and (I-3b), a carbon atom with * indicates the same carbon atom as * in the formula (I-3),
X^{III} represents a structure represented by formula (III-1) or (III-2): 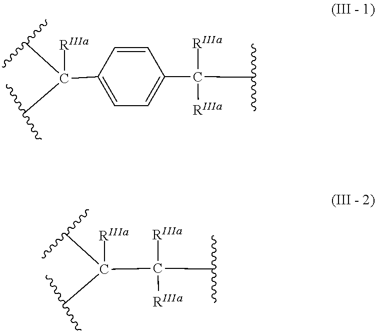
wherein, in the formulae (III-1) and (III-2), R^{IIIa}'s each represent a hydrogen atom or an alkyl group having 1 or 2 carbon atoms, X^{IV} represents a structure represented by formulae (IV-1) to (IV-3): 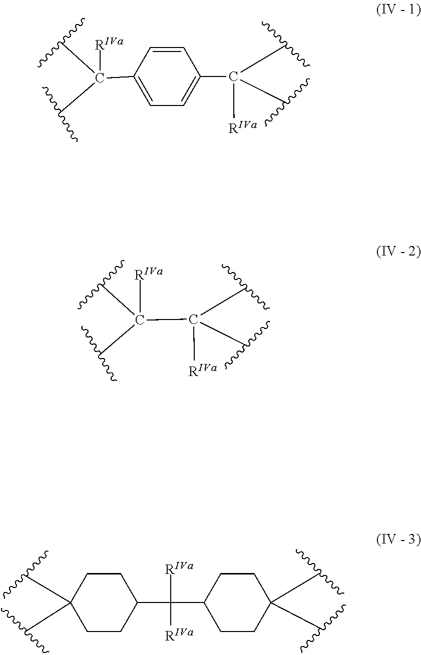
wherein, in the formulae (IV-1) to (IV-3), R^{IVa}'s each represent a hydrogen atom or an alkyl group having 1 or 2 carbon atoms.

5: The photosensitive resin composition according to claim 4, wherein the compound (D) is represented by the formula (I) or (IV).

6: The photosensitive resin composition according to claim 5, wherein the compound (D) is represented by a formula (I) in which the X^{I} has a structure represented by the formula (I-2) or represented by a formula (IV) in which the X^{IV} has a structure represented by the formula (IV-2).

7: The photosensitive resin composition according to claim 1, wherein the compound (D) is a compound having two or more groups that generate the compound (d′) on average in one molecule.

8: The photosensitive resin composition according to claim 1, wherein an amount of the compound (D) in the photosensitive resin composition is 1 to 40 parts by mass with respect to 100 parts by mass of the polymer (A).

9: The photosensitive resin composition according to claim 1, wherein the polymer (A) has a structural unit represented by formula (a1-1): 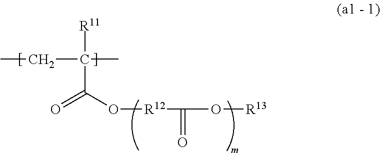
wherein, in the formula (a1-1), R¹¹ is a hydrogen atom, an alkyl group having 1 to 10 carbon atoms, or a group obtained by substituting at least one hydrogen atom in the alkyl group with a halogen atom, an aryl group, a hydroxy group, or an alkoxy group, R¹² is a divalent organic group having 1 to 10 carbon atoms, R¹³ is an acid-dissociable group, and m is an integer of 0 to 10.

10: The photosensitive resin composition according to claim 1, further comprising a compound (E1) which is decomposed by an action of an acid to generate a primary or secondary amine.

11: The photosensitive resin composition according to claim 1, wherein the photoacid generator (B) is a compound represented by formula (b1) or (b2): 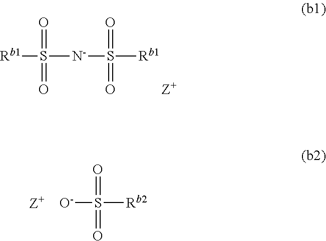
wherein, in the formulae (b1) and (b2), Z⁺ represents an onium cation, R^{b1} and R^{b2} each independently represent an alkyl group having 1 to 10 carbon atoms, a cycloalkyl group, an aryl group, or a group obtained by combining at least two of the alkyl group, the cycloalkyl group, and the aryl group, provided that the alkyl group, the cycloalkyl group and the aryl group are unsubstituted or some or all hydrogen atoms in the alkyl group, the cycloalkyl group and the aryl group are substituted with a fluorine atom, and optionally two R^{b1}'s form a cyclic structure by bonding carbon atoms contained in the R^{b1}'s.

12: The photosensitive resin composition according to claim 1, further comprising an alkali-soluble resin.

13: The photosensitive resin composition according to claim 1, wherein the solvent (C) comprises at least one selected from propylene glycol monomethyl ether acetate, 3-methoxybutyl acetate, and propylene glycol monomethyl ether.

14: A method for producing a resist pattern film, the method comprising:
forming a resin film of the photosensitive resin composition according to claim 1 on a metal film provided on a substrate;
exposing the resin film to light; and
developing the exposed resin film.

15: The method according to claim 14, wherein the resist pattern film has a thickness of 0.5 to 100 μm.

16: A method for producing a plated formed product, the method comprising performing a plating treatment using a resist pattern film produced by the method according to claim 14 as a template.

17: The method according to claim 16, further comprising performing an ashing treatment before performing the plating treatment.

18: The method according to claim 17, wherein the plating treatment is at last one selected from a copper plating treatment, a nickel plating treatment, a tin plating treatment, and a gold plating treatment.

19: The photosensitive resin composition according to claim 3, wherein the compound (D) is represented by any one of formulae (I) to (IV): 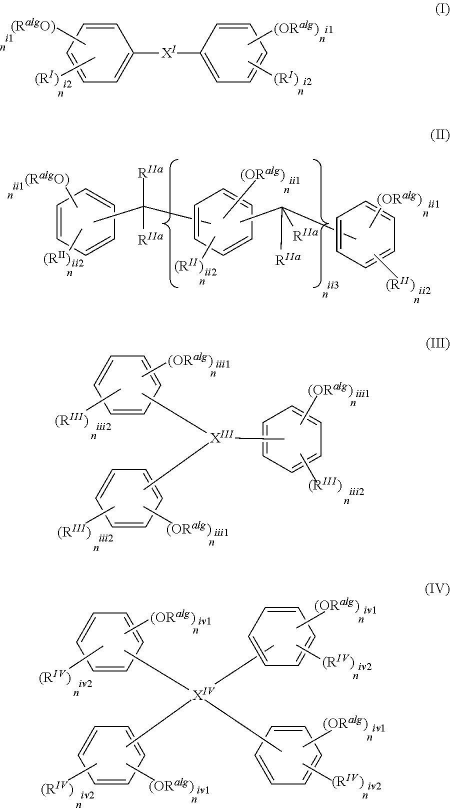
wherein, in the formulae (I) to (IV),
R^{alg}'s each independently represent a hydrogen atom or a group that generates the compound (d′);
R^{I} to R^{IV} each independently represent an alkyl group having 1 to 4 carbon atoms, a cycloalkyl group having 3 to 10 carbon atoms, an aryl group having 6 to 10 carbon atoms, or a halogen atom;
R^{IIa}'s independently represent a hydrogen atom or an alkyl group having 1 or 2 carbon atoms;
nⁱ¹ to n^{iv1} and nⁱⁱ³ each independently represent an integer of 1 or 2;
nⁱ² to n^{iv2} each independently represent an integer of 0 to 2; X¹ represents a single bond or a structure represented by formulae (I-1) to (I-3): 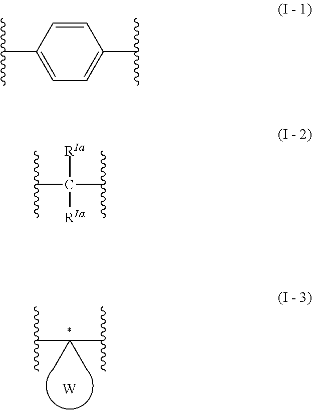
wherein, in the formula (I-2), R^{Ia} represents a hydrogen atom, an alkyl group having 1 to 10 carbon atoms, a cycloalkyl group having 3 to 10 carbon atoms, an aryl group having 6 to 10 carbon atoms, a halogen atom, or a group represented by a formula (I-2a): 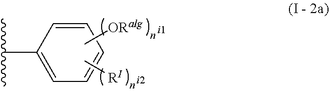
wherein, R^{alg}, R^{I}, nⁱ¹ and nⁱ² are each as defined in the formula (I),
in the formula (I-3), W represents a structure represented by formula (I-3a) or (I-3b): 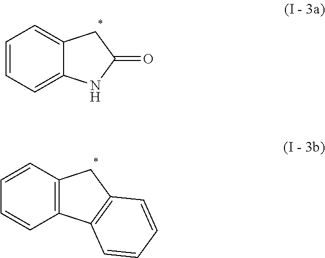
wherein, in the formulae (I-3a) and (I-3b), a carbon atom with * indicates the same carbon atom as * in the formula (I-3),
X^{III} represents a structure represented by a formula (III-1) or (III-2): 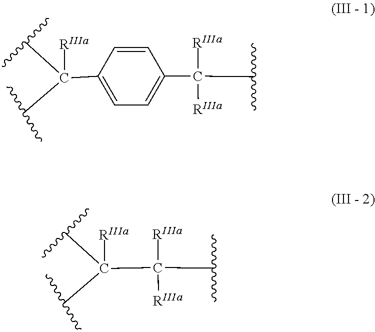
wherein, in the formulae (III-1) and (III-2), R^{IIIa}'s each represent a hydrogen atom or an alkyl group having 1 or 2 carbon atoms,
X^{IV} represents a structure represented by formulae (IV-1) to (IV-3): 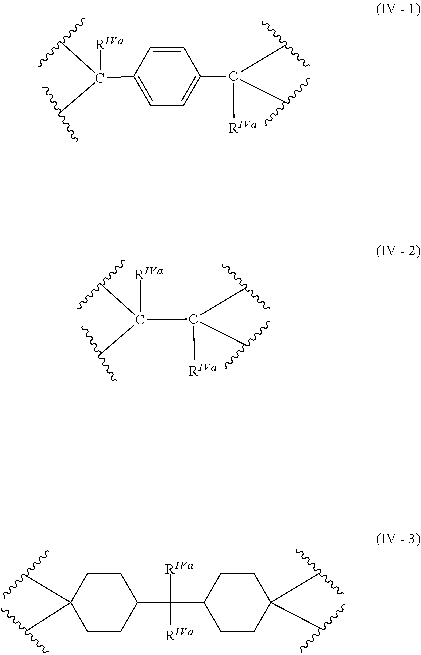
wherein, in the formulae (IV-1) to (IV-3), R^{IVa}'s each represent a hydrogen atom or an alkyl group having 1 or 2 carbon atoms.
